(19) United States
(12) Patent Application Publication
(10) Pub. No.: US 2009/0125015 A1
(43) Pub. Date: May 14, 2009

(54) Electrolysis Hair-Removal Device

(75) Inventor: Olivier Smal, Embourg (BE)

Correspondence Address: REINHART BOERNER VAN DEUREN P.C., 2215 PERRYGREEN WAY, ROCKFORD, IL, 61107, US

(73) Assignee: Faco S.A., Wandre (BE)

(21) Appl. No.: 11/795,215

(22) PCT Filed: Jan. 6, 2006
(86) PCT No.: PCT/BE06/00001

§ 371 Date: Jul. 13, 2007

(30) Foreign Application Priority Data
Jan. 31, 2005 (EP) 05447012.5

(51) Int. Cl. A61B 18/18

(52) U.S. Cl. 606/36; 606/43

(57) ABSTRACT

The invention relates to an electrolysis hair-removal device comprising: a central control unit (4) which is equipped with a microprocessor; and hair-removal patches (8) of variable size, which are adapted to the different parts of the body to be treated, as well as contact patches (10) which can be used to create the electrolysis current on the skin. The invention is characterised in that the device comprises a hair-removal patch identification system which enables the above-mentioned microprocessor to identify said patches and automatically to adapt the duration for which the current is applied in accordance with the intensity selected by the user in order to deliver a constant dose of energy that is specifically associated with the size of the selected hair-removal patch.

### FIELD OF THE INVENTION

The present invention relates to the field of “permanent” or “long-term” hair-removal devices, and in particular to devices for removing hair by electrolysis.

### STATE OF THE ART

Hair removal by electrolysis is well known to the man skilled in the art. This technique consists in transmitting a barely perceptible direct current to the base of the hair follicle, which causes the neutralisation of the dermal papilla feeding hair and severely restricts its regrowth. This type of hair-removal device is directed not only at the visible hair but also at the root to provide “long-term” or even “permanent” hair-removal.

Hair grows in a pocket called a follicle. Depending on the area, the density of follicles varies between about 60 follicles/cm² for the legs and 500 follicles/cm² for the moustache.

Hair grows in cycles: during the growth phase (or anagenic phase), hair is fed by the dermal papilla before it then enters a transition phase (or catagenic phase) and finally a state of rest (or tellogenic phase) during which hair does not grow since it is separated from the dermal papilla and falls out. Afterwards, the follicle comes again into contact with the dermal papilla and a new cycle resumes. Hair needs about three weeks to grow from the base of the follicle to the surface of the skin and thus to be visible. In other words, hair that starts growing will only be visible three weeks later.

From the moment when hair appears at the surface of the skin, the growth period still continues for about another thirteen weeks. For the cosmetic treatment to be effective, hair needs to be treated during the anagenic phase, i.e. when the dermal papillae are still active; it is the essential condition for actually stopping the process of hair growth.

Moreover, hair grows in overlapping cycles: in the same area, some hair is in the growth (anagenic) phase and some other is in the rest (tellogenic) phase; this is why the first results are only obtained after several weeks of application of the device according to the invention to the parts of the body where hair is to be removed.

Devices for permanent or long-term hair removal by electrolysis or any related method are disclosed in particular by patent US-60/397,406 which describes an electric-current generator that transmits a current to a patch stuck to the skin. Documents DE 3 426 141 and U.S. Pat. No. 5,534,003 also describe inventions based on the same principle.

Document U.S. Pat. No. 5,522,814 describes a high-frequency hair-removal method.

Document FR-2 680 965 describes a device and a method for the cosmetic skin treatment aimed at long-term hair removal which consists in inducing a high-frequency electromagnetic current through the skin by means of a conductive gel charged with a treatment product, the electromagnetic current causing this product to penetrate the pores of the skin.

Document FR-2 595 239 discloses a permanent hair-removal device comprising a high-frequency current generator that produces a high-frequency current chopped into batches of phased wave that can be applied to the skin.

The problem encountered with all the devices known in the prior art lies in the fact that the user himself can choose both the intensity and the duration, hence the application of energy of the electrolysis current. The energy to be supplied must be adapted to the size of the surfaces of the body to be treated and to their sensitivity. This often led to inappropriate use of this type of device, possibly leading to irritation of the skin.

Document US 2004/0039403 A1 discloses a battery-operated hair remover based on the principle of low-frequency electrolysis with independently-fed patches.

Document U.S. Pat. No. 5,906,614 discloses a system for the ablation of cardiac tissue by means of energetic elements based on catheters transmitting their energy to the cardiac tissues and provided with precise temperature probes. The system comprises a device for controlling the temperatures at the level of the ablation catheter, which is extremely complex and ill-suited for managing and recognising hair-removal patches stuck to the skin.

It was therefore necessary to solve this problem by proposing a device capable of administering an energy dose that is precise and constant depending on the surfaces of the patches used and it was therefore necessary to develop a device capable of identifying patches of different sizes in order to allow precise and constant dosage of the energy administered to a given area.

### AIMS OF THE INVENTION

The present invention aims to provide a device for removing hair by electrolysis comprising a central control unit provided with a microprocessor and a set of (hair-removal and contact) patches capable of applying precise current intensities for precise durations so as to apply constant doses of energy per identified patch. To achieve this, the invention proposes a system for recognising the patches that allows the application of targeted doses for each type of use.

### SUMMARY OF THE INVENTION

The present invention discloses a device for hair removal by electrolysis comprising a central control unit provided with a microprocessor and hair-removal patches of variable size adapted to the different parts of the body to be treated as well as contact patches allowing to create an electrolysis current on the skin, characterised in that said device comprises a system for identifying the hair-removal patches allowing the microprocessor of the central control unit to identify them and to regulate the duration of application of the current depending on the intensity selected by the user so as to administer a dose of energy that is specifically tailored to the size of the hair-removal patch used.

According to particular embodiments of the application, the invention comprises one or several of the following characteristics:
the hair-removal device also comprises a precision stylet,
said precision stylet comprises several interchangeable application heads,
the central unit comprises an visual system with a display of various parts of the body allowing the user to choose the parts to be treated,
the visual system comprises a timer displaying the remaining treatment duration,
said identification device (5,6,7) comprises an oscillating circuit,
said identification device (5,6,7) comprises resistors on the connectors of the patches (8) or of the stylet,
said identification system comprises a resistor (5,6,7) located on the patch (8) or on the stylet (9) to be recognised as well as a capacitor located on the central control unit, the microprocessor of said unit (4) allowing to recognize the frequency of the oscillating circuit and to adjust the energy supplied by the device depending on the size and function given to the chosen patch (8).

The invention also discloses the use of the device according to Claim 1 for long-term hair removal.

### BRIEF DESCRIPTION OF THE FIGURES

FIG. 1 represents the connection layout of the patches on the central control unit.

FIG. 2 represents the connection layout of the precision stylet on the central control unit.

FIG. 3 represents a view of the whole device with the central unit, its patches and the precision stylet.

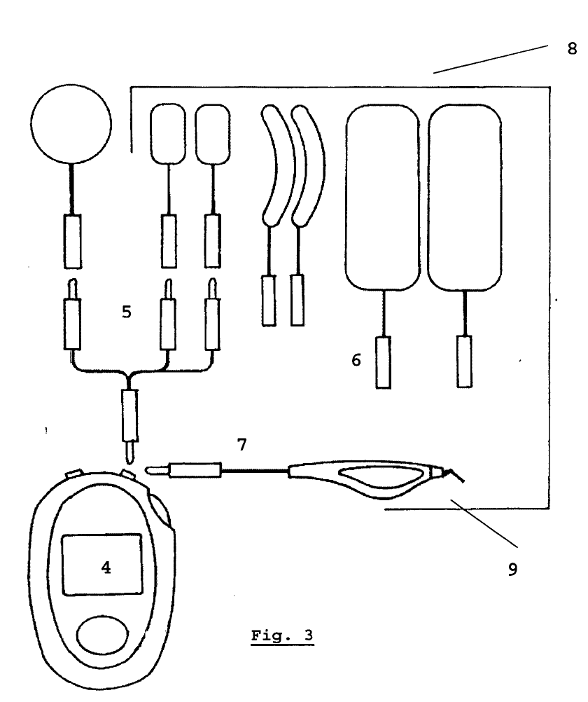

### DETAILED DESCRIPTION OF THE INVENTION

The device for electrolysis hair removal comprises a central control unit 4 provided with a microprocessor, various patches 8 of different sizes adapted to the application areas (face, legs, sensitive areas) and contact patches 10 that allow to close the circuit and transmit the electrolysis current by the skin. The assembly also comprises a precision stylet 9 with several interchangeable heads.

The electrolysis hair-removal device of the present invention is provided with an automatic device for identifying the patches. The central control unit 4 will therefore “recognise” the patches 8 or the stylet 9 which will be connected and the areas to be treated will be displayed on the model on the screen of this unit 4. The central control unit 4 allows to adjust the intensity of the current depending on the sensitivity of the skin. Then the device of the invention will automatically adapt the treatment duration. The central unit 4 comprises a timer that displays on the screen the remaining duration of treatment.

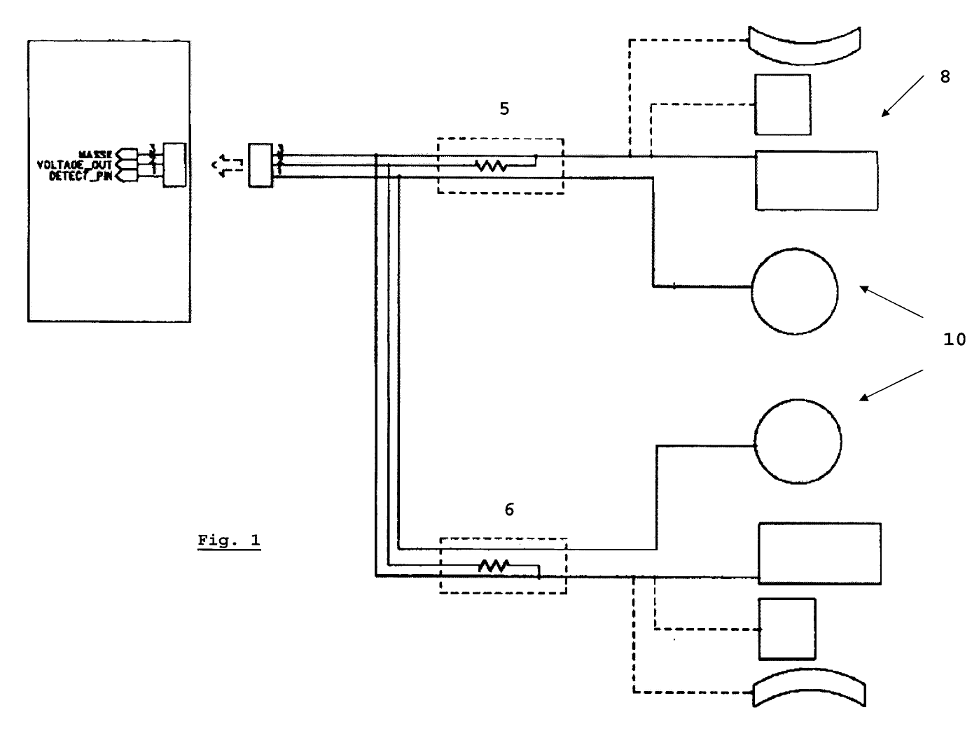

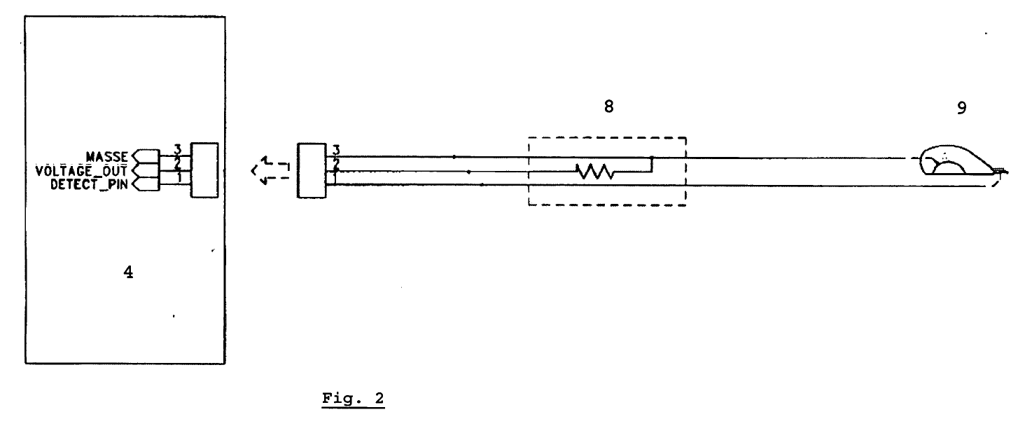

In order to identify the patches 8 or the stylet 9 used, their connectors are provided with an electric resistor (5,6,7), which allows the device, by means of an oscillating circuit, to detect which type of patch is being used (FIGS. 1 and 2).

The circuit comprises a resistor (5,6,7) located in the patch or in the stylet to be recognised and a capacitor located on the central control unit. The microprocessor of this unit 4 recognises the frequency of the oscillating circuit and adapts the energy supplied by the device depending on the size and function given to the chosen patch 8.

The connection layout of the patches 8 to the central unit 4 is shown in FIG. 1. In order to recognise the frequency of the oscillating circuit and to adjust the energy supplied, two electric wires 2 and 3 are connected to the patches 8 or to the stylet 9 (see FIG. 2) so as to close the oscillating circuit. The wire 3 is used to close the circuit via the user's skin and the contact patch 10. In order to supply constant energy to the user, the microprocessor adjusts the time depending on the current intensity chosen by the user. In this way, whatever the changes in intensity caused by the user during the treatment period, the total amount of current over the operating period remains constant and the device thus supplies a constant dose of energy for each type of patch.

The users of this type of device usually have very different sensitivity levels relative to the number of milliamperes applied to their skin. This is why everyone adjusts the intensity of the current to their own comfort level. Then the control unit 4 manages the duration.

The following table shows some examples of different resistors allowing to recognise the patches:

| Patch and stylet | Resistor |
|---|---|
| Patch for the legs | 430,000 Ohm |
| Patch for sensitive parts | 150,000 Ohm |
| Patch for the face | 47,000 Ohm |
| Precision stylet | 15,000 Ohm |

In each operating mode, the intensity of current multiplied by the time is a constant.

| | | | Current × time = |
|---|---|---|---|
| Mode | Current (mA) | Time (s) | energy supplied |
| Stylet | 0.2 to 0.5 mA | 50 to 20 | 10 |
| Face | 0.5 to 2 mA | 240 to 60 | 120 |
| Sensitive | 1 to 4 mA | 240 to 60 | 240 |
| Legs | 2.5 to 10 mA | 240 to 60 | 600 |

The system thus allows to supply an amount of energy that is always identical whatever the intensity chosen by means of the control of the energy supplied; this amount is defined depending on the patch connected by its identification system and on the adjustment of the application duration.

The precision stylet 9 is used to treat precise areas and it may be provided with four different heads depending on the area to be treated: the wide head is used for the moustache and the finer head is used for isolated hair. The use of a conductive gel may improve the conductivity of the precision stylet or that of the patches.

### Key

1 Wire connected to the contact patch
2 Wire connected to the resistor of the oscillating circuit
3 Detection wire connected to the patches for electrolysis hair-removal
4. Central control unit
5,6,7 Transfer socket (recognition resistors)
8 Patches for electrolysis hair removal
9 Precision stylet
10 Contact patches closing the circuit

1. Device for electrolysis hair removal comprising a central control unit (4) provided with a microprocessor and hair-removal patches (8) of variable size adapted to the different parts of the body to be treated as well as contact patches (10) allowing to create an electrolysis current on the skin, characterised in that said device comprises a system for identifying hair-removal patches (8) allowing the microprocessor of the central control unit (4) to identify them and to automatically adjust the application duration of the current depending on the intensity chosen by the user in order to supply a constant dose of energy specifically tailored to the size of the hair-removal patch (8) used.

2. Hair-removal device according to claim 1, characterised in that it also comprises a precision stylet (9).

3. Hair-removal device according to claim 2, characterised in that said precision stylet comprises several interchangeable application heads.

4. Hair-removal device according to claim 1, characterised in that the central unit (4) comprises a visual system displaying different parts of the body on a model allowing the user to choose the parts of the body to be treated.

5. Hair-removal device according to claim 4, characterised in that the visual system comprises a timer that displays the remaining treatment duration.

6. Hair-removal device according to claim 1, characterised in that said identification device (5,6,7) comprises an oscillating circuit.

7. Hair-removal device according to claim 1, characterised in that said identification device (5,6,7) comprises resistors on the connectors of the patches (8) or of the stylet (9).

8. Hair-removal device according to claim 1, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

9. Use of the device according to claim 1 for long-term hair-removal.

10. Hair-removal device according to claim 2, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

11. Hair-removal device according to claim 3, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

12. Hair-removal device according to claim 4, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

13. Hair-removal device according to claim 5, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

14. Hair-removal device according to claim 6, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).

15. Hair-removal device according to claim 7, characterised in that said identification system comprises a resistor (5,6,7) located in the patch (8) or the stylet (9) to be recognised as well as one capacitor located on the central control unit, the microprocessor of said unit (4) allowing to recognise the frequency of the oscillating circuit and to adjust the energy supplied by the device depending on the size and function given to the chosen patch (8).
